(19) United States
(12) Patent Application Publication
(10) Pub. No.: US 2025/0035553 A1
(43) Pub. Date: Jan. 30, 2025

(54) METHODS OF MULTIPLEXED ANALYTE CHARACTERIZATION

(71) Applicant: Waters Technologies Corporation, Milford, MA (US)

(72) Inventor: Kevin Wyndham, Worcester, MA (US)

(73) Assignee: Waters Technologies Corporation, Milford, MA (US)

(21) Appl. No.: 18/785,572

(22) Filed: Jul. 26, 2024

Related U.S. Application Data
(60) Provisional application No. 63/516,192, filed on Jul. 28, 2023.

(51) Int. Cl. G01N 21/64; G01N 33/68

(52) U.S. Cl. CPC G01N 21/643; G01N 33/6854; G01N 2021/6439; G01N 2440/38

(57) ABSTRACT

Disclosed herein are methods for the multiplexed characterization of analytes using dye-tagged affinity molecules. The DTAMs, useful in conjunction with liquid chromatography systems, allow for the parallel quantitation and characterization of attributes of one or more target analytes from complex solutions.

### CROSS-REFERENCE TO RELATED APPLICATIONS

This application claims the benefit of U.S. Provisional Application No. 63/516,192 filed on Jul. 28, 2023, the entirety of which is incorporated herein by reference.

### FIELD OF INVENTION

The present disclosure relates generally to the characterization of target analytes using analytical methods. More specifically, the disclosure pertains to the characterization of multiple attributes of a target analyte in parallel.

### BACKGROUND

The ability to characterize compounds by key attributes, such as size, mass, charge, and/or quantity is foundational to the pharmaceutical, chemical, and life sciences industries. Yet analytical methods to do so often only generate data for a single attribute, requiring multiple, distinct assays to collect the necessary data. This approach is burdensome with respect to sample preparation, reagent expenses, and overall time.

Liquid chromatography is often utilized in the aforementioned methods, as it can be customized by both column type and downstream detector, affording quality data regarding a particular attribute. But liquid chromatography applications are limited in this same respect, requiring separate samples and specific column/detector combinations for a particular analysis (i.e., a size-exclusion column with a multi-angle light scattering detector or a reversed-phase column with a mass spectrometry detector).

Accordingly, there exists a need in the art for methods that permit multiplexed characterization of analytes using liquid chromatography.

### SUMMARY OF INVENTION

In one aspect disclosed herein is a method for the simultaneous quantification and characterization of analytes in a composition, the method comprising a) selecting at least one dye-tagged affinity molecule (DTAM) of the formula D₁₋₈-L₀₋₈-M, wherein D represents an ultraviolet (UV) or fluorescent dye molecule, -L-represents an optionally substituted aliphatic, aromatic, heterocyclic, polyethylene glycol, carbohydrate, or peptide linker, and M is an affinity group; b) adding the at least one DTAM to a sample comprising at least one target analyte; c) injecting the sample into a liquid chromatographic system comprising at least one detector; and d) measuring at least one attribute of the at least one target analyte with the at least detector. In some embodiments, D is 1 and L is 1.

In another aspect, disclosed herein is a method for the simultaneous quantification and characterization of analytes in a composition, the method comprising: a) selecting at least one dye-tagged affinity molecule (DTAM) of the formula L^{A}₀₋₈-D₁₋₈-L₀₋₈-M-L^{B}₀₋₈, wherein -L-. L^{A} and L^{B} represent optionally substituted aliphatic, aromatic, heterocyclic, polyethylene glycol, carbohydrate, or peptide linkers, and M is an affinity group; b) adding the at least one DTAM to a sample comprising at least one target analyte; c) injecting the sample into a liquid chromatographic system comprising at least one detector; and d) measuring at least one attribute of the at least one target analyte with the at least one detector. In some embodiments, D is 1, L is 1, and L^{A} and/or L^{B} are 1. In some embodiments, L^{A} and/or L^{B} are absent (i.e., are 0).

In some embodiments, the detector is selected from the group consisting of: UV, fluorescence, multi-angle light scattering, asymmetric flow field-flow fractionation, evaporative light scattering, refractive index, conductivity, charged aerosol, a charge detection mass spectrometry, or mass spectrometry detector. In some embodiments, the liquid chromatographic system comprises a size-exclusion column, reversed phase column, hydrophilic interaction column, hydrophobic interaction column, ion exchange column, chiral column or combinations thereof. In some embodiments, the affinity group is selected from the group consisting of: a polyclonal antibody, a monoclonal antibody, a fusion protein, a bispecific antibody, a chimeric antibody, a monobody, a nanobody, a camelid, an affimer, an affibody, an aptamer, a cyclodextrin, a peptide, DNA, RNA, a lectin, a chelating agent, a molecular imprinted polymer, or combinations thereof. In some embodiments, the target analyte is a small molecule, a peptide, a protein, an antibody, a hormone, a pesticide, a toxin, and/or nucleic acids.

In one aspect of the methods disclosed herein, the target analyte in the sample and the DTAM are at equivalent concentrations. In some embodiments, the DTAM is at a higher concentration than the target analyte in the sample. In some embodiments, the attribute being measured is quantity, size, charge, post-translational modification, glycan concentration, and/or aggregation.

In some embodiments of the methods disclosed herein, more than one attribute is measured in parallel. In some embodiments, the attributes measured in parallel are quantity, glycan concentration, and size. In some embodiments, the attributes measured in parallel are antibody quantity and mannose, fucose, and/or a sialylated glycan concentration.

In one aspect, disclosed herein are methods wherein two to six DTAMs are selected, wherein each of the two to six DTAMs have different emission wavelengths. In some embodiments, two DTAMs are selected, wherein the affinity groups of each of the two DTAMs are an antibody. In some embodiments, three DTAMs are selected, wherein the affinity groups of each of the three DTAMs are an antibody. In some embodiments, the attributes measured in parallel are antibody quantity, mannose, fucose, and/or a sialylated glycan concentration, and size. In some embodiments, the attributes measured in parallel are antibody quantity, mannose, fucose, and/or a sialylated glycan concentration, and charge. In some embodiments, the more than one DTAMs have affinity groups that bind to the same target analyte. In some embodiments, the more than one DTAMs have affinity groups that bind to unique target analytes.

### BRIEF DESCRIPTION OF THE DRAWINGS

FIG. 1 is a graphical illustration of a method of characterizing multiple attributes in parallel in accordance with an embodiment of the technology.

FIG. 2 is a graphical overview of a liquid chromatography system with two detectors connected in series in accordance with an embodiment of the technology.

FIG. 3A, FIG. 3B and FIG. 3C provide a representative depiction of fluorescence chromatograms resulting from a multiplexed assay using two DTAMs according to an embodiment of the technology.

FIG. 4A, FIG. 4B, and FIG. 4C provide a representative depiction of fluorescence chromatograms resulting from a multiplexed assay using two DTAMs according to an embodiment of the technology.

FIG. 5 provides an exemplary synthesis of a DTAM according to an embodiment of the technology.

FIG. 6 provides chromatograms of DTAM and IgG samples analyzed via size exclusion chromatography.

FIG. 7 provides a comparison of chromatograms of a DTAM and IgG sample (top) as compared to a known standard (bottom).

### DETAILED DESCRIPTION

In order that the technology may be more readily understood, certain terms are first defined. In addition, it should be noted that whenever a value or range of values of a parameter are recited, it is intended that values and ranges intermediate to the recited values are also part of this disclosure. It is also to be noted that as used herein, and in the claims, the singular forms “a” and “the” include plural references unless the context clearly indicates otherwise.

### Definitions

As used herein, the term “attribute” refers to a quantity of or a secondary characteristic of a compound or compounds. Secondary characteristics can include, but are not limited to, size, charge, post-translational modification, glycan concentration, and/or aggregation. A quantity of a compound or compounds refers to the amount or concentration of said compound or compounds. The quantity can be an absolute quantity or a relative quantity of the compound as compared to other compound(s) in the sample.

The term “dye-tagged affinity molecule” or “DTAM” refers to a molecule comprising a fluorescent or ultraviolet dye and an affinity group optionally connected by a linker. DTAMs of the present technology are represented by, for example, the formula D₁₋₈-L₀₋₈-M, wherein D is the fluorescent or ultraviolet dye, L is the linker, and M is the affinity group.

As used herein, the term “affinity group” refers to a compound comprising a moiety that specifically binds to a target analyte. For example, an affinity group could be an antibody that specifically binds to its target antigen or a lectin that specifically binds to a target glycan. As one or ordinary skill in the art will understand, the target analyte is dependent on the affinity group moiety. In one embodiment of the technology, the affinity group is an antibody that binds to IgG. In another embodiment of the technology, the affinity group is a lectin that binds to mannose. In some embodiments, the target analyte of each affinity group may be present on the same compound (i.e., a glycosylated antibody).

The term “antibody,” as used herein refers to an immunoglobin molecule that specifically binds to, or is immunologically reactive with, a particular antigen. This includes polyclonal, monoclonal, genetically engineering, and otherwise modified forms of antibodies, including but not limited to chimeric antibodies, camelids, monobodies, humanized antibodies, and heteroconjugate antibodies (e.g., bi-, tri-, and quad-specific antibodies, diabodies). Unless otherwise indicated, the term “monoclonal antibody” is meant to include both intact molecules as well as antibody fragments that are capable of specifically binding to a target protein.

As used herein, the term “specifically binds” refers to the ability of an affinity group to recognize and bind a specific analyte rather than to analytes generally. By way of example, an antibody that specifically binds to antigen A would, in a sample comprising a mixture of antigens, preferentially bind to antigen A.

As used herein, the terms “measured in parallel”, “measured in tandem”, and “measured simultaneously” or more generally processes described as being done “in parallel”, “simultaneously”, or “in tandem” refers to the characterization of more than one attribute, and/or more than one target analyte in a single assay. For example, in one embodiment of the technology, the quantity of target analyte and the concentration of mannose are characterized in a single assay, and thus measured in parallel. In another embodiment of the invention, the quantity of a target analyte, concentration of mannose, and size of the target analyte are characterized in a single assay, and thus measured in parallel.

As used herein, the term “multiplex” or “multiplexed” refers to an assay designed to characterize two or more target analytes in parallel.

Unless defined otherwise, all technical and scientific terms used herein have the same meaning as commonly understood by one or ordinary skill in the art to which this disclosure pertains.

### Dye-Tagged Affinity Molecules

In one aspect, disclosed herein are dye-tagged affinity molecules (DTAMs) designed to be used in the characterization of target analytes. DTAMs of the present technology comprise a fluorescent or UV dye and an affinity group, optionally connected by a linker. As such, the DTAMs of the instant technology specifically bind to a target analyte via the affinity group and confer by association a fluorescent or UV signal. For example, a DTAM comprising an antibody affinity group will bind to its target antigen, and the antigen will be associated with the fluorescent or UV signal of the DTAM. Consequently, the fluorescent or UV signal of bound DTAMs as measured by an appropriate detector is directly related to the bound target analyte.

When coupled with liquid chromatography, the DTAMs of the instant technology and the methods disclosed herein permit the characterization of multiple attributes of a target analyte. Liquid chromatography enables the separation of compounds by certain parameters, including size (e.g., size-exclusion chromatography), charge (e.g., ion-exchange chromatography), and polarity (e.g., normal-phase, reversed-phase, and hydrophobic interaction chromatography), amongst others. Liquid chromatography systems can further be coupled to detectors, for example fluorescence detectors, ultraviolet detectors, and/or mass spectrometry detectors.

By combining the DTAMs with the separation and detection capabilities of liquid chromatography systems, multiple attributes of a target analyte can be determined in a single assay. Further, multiple DTAMs with fluorescent dyes can be designed such that the emission wavelengths of the dyes do not overlap. Thus, the DTAMs can be added to a single sample, allowing for the multiplexed characterization of more than one target analyte using a single assay.

Accordingly, disclosed herein are DTAMs of the formula D₁₋₈-L₀₋₈-M, wherein:
D represents an ultraviolet (UV) or a fluorescent dye molecule;
-L-represents an optionally substituted linker;
and M is an affinity group.

In other embodiments, disclosed herein are DTAMs of the formula L^{A}₀₋₈-D₁₋₈-L₀₋₈-M-L^{B}₀₋₈, wherein:
D represents an ultraviolet (UV) or a fluorescent dye molecule;
-L-, L^{A}, and L^{B} represent an optionally substituted linker;
and M is an affinity group.

In some embodiments, L^{A} and/or L^{B} may be absent, i.e., L^{A} and/or L^{B} are equal to 0.

A number of dyes are suitable for use with the present technology. In one embodiment, the dye is fluorescein-5-isothiocyanate (5-FITC, ThermoFisher Scientific). In some embodiments, the dye is tetramethylrhodamine-5-isothiocyanate (5-TRITC), rhodamine-B isothiocyanate, or NIR-797 isothiocyanate. In some embodiments the dye is Alexa Fluor® 350 (ThermoFisher Scientific) or Alexa Fluor® 650 (ThermoFisher Scientific). In some embodiments, the dye is selected from the group consisting of: R-Phycoerthryin (RPE; ThermoFisher Scientific), Texas Red (ThermoFisher Scientific), the DyLight® series (ThermoFisher Scientific), the ATTOR series (Sigma-Aldrich), the Alexa Fluor® series (ThermoFisher Scientific), the Brilliant Ultra Violet series (ThermoFisher Scientific), the Brilliant Violet series (ThermoFisher Scientific), the eFluor® series (ThermoFisher Scientific), cyan fluorescent protein (CFP; ThermoFisher Scientific), green fluorescent protein (GFP; ThermoFisher Scientific), red fluorescent protein (RFP; ThermoFisher Scientific), the NovaFluor® Blue series (ThermoFisher Scientific), the NovaFluor® Yellow series (ThermoFisher Scientific), BODIPY® fluorescent dye (ThermoFisher Scientific), Oregon Green 488 (ThermoFisher Scientific), the Pacific® series (ThermoFisher scientific), allophycocyanin (APC; ThermoFisher Scientific), PE-Cyanine7 (ThermoFisher Scientific), PE-cFluor 610 (ThermoFisher Scientific), PerCP-Cyanine5.5 (ThermoFisher Scientific), PerCP-eFluor 710 (ThermoFisher Scientific), R-phycoerythrin (R-PE; ThermoFisher Scientific), and the Super Bright® series (ThermoFisher Scientific).

A dye that is detectable with a UV or fluorescence detector is suitable for use in the methods disclosed herein, however certain dyes may not be amenable for particular chromatography columns. Without wishing to be bound by any particular theory, certain dyes may result in reduced separation performance due to undesirable hydrophobic or ionic interactions with the stationary phase of the chromatography column. The retention time of an unbound DTAM can be determined on a column as compared to one or more calibration standards. If the retention time of the DTAM significantly deviates from the expected retention time, it can be indicative of undesirable interactions with the stationary phase. For illustrative purposes, an assay for assessing a DTAM for retention on a size-exclusion chromatography column is described in Example 6.

In some embodiments, the dye molecule of the DTAM does not have a substantial change in emission intensity and/or wavelength when bound to its target analyte versus unbound. In some embodiments, the dye molecule of the DTAM does have a change in emission intensity and/or wavelength when bound to its target analyte versus unbound. To determine this, the relative change in emission intensity and/or wavelength for both bound versus unbound states can be measured. Appropriate placement of the dye molecule conjugated to the affinity group or the use of a suitable linker can minimize a change in emission intensity and/or wavelength. In embodiments wherein the dye molecule does have a change in emission intensity and/or wavelength when bound to its target analyte versus unbound, the detection method is adjusted to account for the change.

Further, a number of affinity groups are suitable for use with the present technology. In one embodiment, the affinity group is an antibody. In one embodiment, the affinity group is a lectin. In some embodiments, the affinity group is selected from the group consisting of: a fusion protein, an affimer, an aptamer, an affibody, cyclodextrin, a peptide, nucleic acids, affinity tags, proteo-nucleic complexes, chelating agents, and molecular imprinted polymers. Provided the affinity group specifically binds to a target analyte, it is suitable for use in conjunction with the present technology.

In some embodiments, the affinity molecule is Protein A, Protein G, Protein A/G, Protein L, an anti-AAV antibody, or an anti-Host-cell protein antibody (HCPs).

In some embodiments, the affinity group is directly conjugated to the dye. In other embodiments, the affinity group is conjugated to the dye via a linker. Methods of conjugating compounds are well established and known in the art (see, e.g., Bioconjugate Techniques; Greg T. Hermanson, Academic Press, 2013).

Linkers suitable for use in the present technology include, but are not limited to, aliphatic, aromatic, heterocyclic, polyethylene glycol, carbohydrate, oligosiloxane, polyhedral oligomeric silsesquioxane, or peptide linkers. Linkers of the present technology can be optionally substituted. The term “optionally substituted” indicates that a group may or may not be further substituted with one or more groups selected from, but not limited to, hydroxyl, alkyl, alkoxy, alkoxycarbonyl, alkenyl, alkonyloxy, alkynyl, amino, aminoacyl, thio, arylalkyl, arylalkyoxy, aryl, aryloxy, acylamino, carboxy, cyano, halogen, nitro, sulfo, phosphono, phosphorylamino, phosphinyl, hetroaryl, hetroaryloxy, hetrocyclyl, heterocycloxy, trihalomethyl, pentafluoroethyl, trifluoromethoxy, or difluoromethoxy. Additionally or alternatively, the linkers may be optionally substituted with 1, 2, 3, 4, 5, 6 or more deuterium atoms.

In some embodiments, a single DTAM molecule comprises 1, 2, 3, 4, 5, 6, 7, or 8 dye molecules. In some embodiments, a single DTAM molecule comprises 0, 1, 2, 3, 4, 5, 6, 7, or 8 linkers. In one embodiment, a single DTAM molecule comprises 1 dye molecule and no linker. In one embodiment, a single DTAM molecule comprises 1 dye molecule and 1 linker.

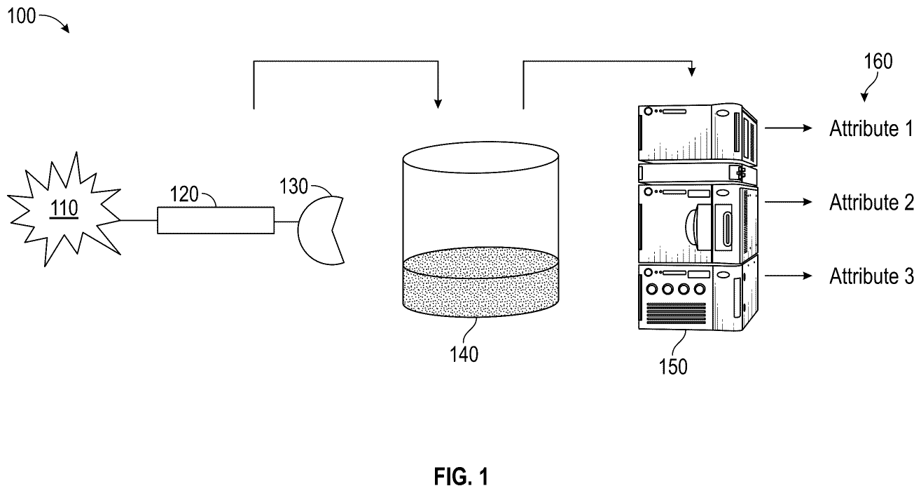

FIG. 1 provides a representative DTAM molecule (100), comprising a dye molecule (110), a linker (120), and an affinity group (130). In some embodiments, linker (120) is absent. In some embodiments, dye molecule (110) is a fluorescent dye. In some embodiments, affinity group (130) is an antibody. In some embodiments, affinity group (130) is a lectin.

### Methods of Use

The DTAMs disclosed herein can be utilized in methods for the parallel characterization of multiple attributes of a target analyte. The methods disclosed herein rely on a combination of the analytical separation afforded by liquid chromatography in conjunction with the fluorescent property of the DTAM-bound target analyte.

Accordingly, in one aspect disclosed herein is a method of characterizing multiple attributes of a target analyte in parallel. The methods disclosed herein utilize a DTAM of the present technology and a liquid chromatography system comprising one or more detectors. As one of skill in the art will appreciate, the detectors used in conjunction with the liquid chromatography system will determine, in part, the attributes that are characterized. Any detector designed for use in conjunction with a liquid chromatography system is suited for use in the present technology, including but not limited to UV, fluorescence (FL), multi-angle light scattering (MALS), asymmetric flow field-flow fractionation (AF4), evaporative light scattering (ELS), refractive index (RI), conductivity, charged aerosol (CAD), or mass spectrometry (MS) detectors. At a minimum, the method requires a detector that can measure the dye of the DTAM. That is, if a DTAM comprises a fluorescent dye, a FL detector is required. If a DTAM comprises a UV dye, a UV detector is required. To allow for multiplexed assays, wherein more than one target analyte is characterized simultaneously, the fluorescence detector must be capable of detecting multiple excitation/emission wavelengths in tandem. More than one detector can be utilized by connecting the detectors in series.

Additionally, the liquid chromatography system can optionally be equipped with a column that provides an initial degree of analyte separation prior to detection. The selected column can separate analytes in a sample by size, charge, and/or polarity, alone or in combination (mixed mode). Columns suitable for use with the methods disclosed herein are known in the art, and can include columns suitable for size-exclusion, normal phase, reversed phase, hydrophobic interaction, hydrophilic interaction, and ion exchange. In some embodiments, the liquid chromatography system is equipped with one column. In some embodiments, the liquid chromatography system is equipped with one, two, three, or more columns. In some embodiments, the liquid chromatography system is equipped with a size-exclusion column.

In some embodiments, the methods can be performed in the absence of a liquid chromatography system. In this respect, the fluorescence or UV can be measured using a fluorimeter or a UV spectrophotometer.

FIG. 1 provides an overview of an illustrative method according to some embodiments of the technology. The DTAM molecule (100) comprises an affinity group (130), connected with a linker (120), and a dye molecule (110). DTAM (100) is added to a sample (140), which includes a target analyte to which affinity group (130) specifically binds. Sample (140) can be a biological, chemical, and/or environmental sample, and can be homogenous or heterogenous in composition. Sample (140) is then flowed through a liquid chromatography system (150) comprising at least one detector, which measures one or more attributes (160). In some embodiments, an internal standard is spiked into the sample to allow for accurate quantitation of the target analyte. In some embodiments, a separately run calibration curve is used to allow for accurate quantitation of the target analyte. Methods of using internal standards and/or calibration curves are well-established in the art.

In some embodiments of the methods disclosed herein, the concentration of DTAM is at equivalent or higher concentrations as compared to the target analyte in the sample. As a control, unbound DTAM at known concentrations can be analyzed to determine the elution time and peak intensity for a given concentration of DTAM. For analysis of a target analyte in a sample, the peak of the unbound DTAM can be compared to the separately run control at the same concentration, and changes in peak intensity can inform changes in DTAM concentration.

While FIG. 1 provides an illustrative method, it is understood that variations of said method that differ by DTAM composition and liquid chromatography system fall within the scope of the instant disclosure. For example, in some embodiments, two, three, four, five, six, or more DTAMs are used in a single assay. In some embodiments, two DTAMs are used in a single assay. In some embodiments, three DTAMs are used in a single assay. The use of multiple DTAMs results in a multiplexed assay, allowing for the characterization of more than one target analyte simultaneously. For instance, two DTAMs, with affinity groups that specifically bind to unique analytes, allows for the characterization of two distinct analytes. Three DTAMs, with affinity groups that specifically bind to unique analytes, allows for the characterization of three distinct analytes, and so on. In some embodiments, the two more DTAMs specifically bind to different components of the same target analyte. For example, a DTAM that binds to an antibody (IgG) and a DTAM that binds to fucose, if used in the same assay, would both be bound to any fucosylated IgG antibody. Similarly, a DTAM that binds to an antibody (IgG) and a DTAM that binds to mannose, if used in the same assay, would both be bound to any mannosylated IgG antibody.

In multiplexed assays, the ability to differentiate the signal contributed by the DTAM requires that they be “compatible dyes.” As used herein “compatible dyes” refers to two or more dyes that do not overlap in, or are otherwise distinguishable from each other by, their respective emission spectra. For example, a first fluorescent dye (e.g., Alexa Fluor® 350; ThermoFisher Scientific) with an emission wavelength of 442 nm and a second fluorescent dye (e.g., Alexa Fluor® 680; ThermoFisher Scientific) with an emission wavelength of 702 nm are compatible dyes. In contrast, a first fluorescent dye (e.g., Alexa Fluor® 350; ThermoFisher Scientific) with an emission wavelength of 442 nm and a second fluorescent dye (e.g., Alexa Fluor® 405; ThermoFisher Scientific) with an emission wavelength of 421 nm are “incompatible dyes.” “Incompatible dyes” as used herein refers to two or more dyes that overlap in, or are otherwise not distinguishable from each other by, their respective emission spectra.

Fluorescent dyes have unique excitation/emission wavelengths, and methods of determining compatible dyes are well understood in the art. Thus, a person of ordinary skill in the art would be readily capable of determining compatible dyes useful for preparing or selecting DTAMs suitable for use in the multiplexed methods disclosed herein.

The methods disclosed herein thus provide the characterization of one or more attributes for one or more target analytes in a single assay. The attributes characterized are dependent on the detectors utilized. In some embodiments, a UV detector is used. In some embodiments, a fluorescence detector is used. In some embodiments, both a UV detector and a fluorescence detector are used, connected in series. In some embodiments a fluorescence detector is connected in series with any detector suitable for use in liquid chromatography systems. In some embodiments, a UV detector is connected in series with any detector suitable for use in liquid chromatography systems. In some embodiments, a UV detector and a fluorescence detector connected in series are further connected in series to any detector suitable for use in liquid chromatography systems. In other embodiments, a fluorimeter is used to quantify the fluorescence. In some embodiments, a spectrophotometer is used.

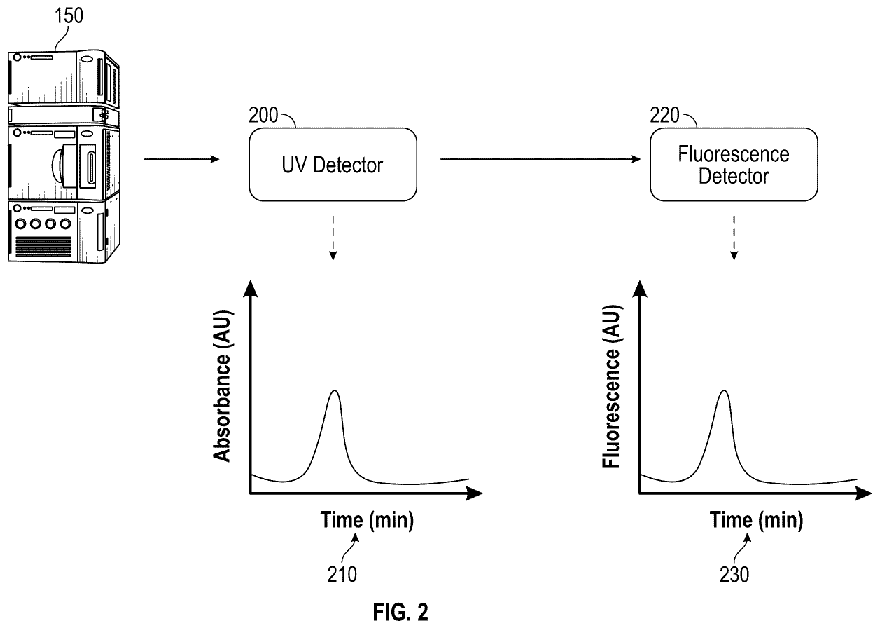

FIG. 2 provides an illustrative overview of a liquid chromatography system with a UV and fluorescence detector connected in series for use in conjunction with the methods disclosed herein. Liquid chromatography system (150) comprises a UV detector (200) and a fluorescence detector (220) connected in series. The UV detector (200) produces a UV chromatogram (210) and the fluorescence detector (220) produces a fluorescence chromatogram (230).

### Characterization of Attributes

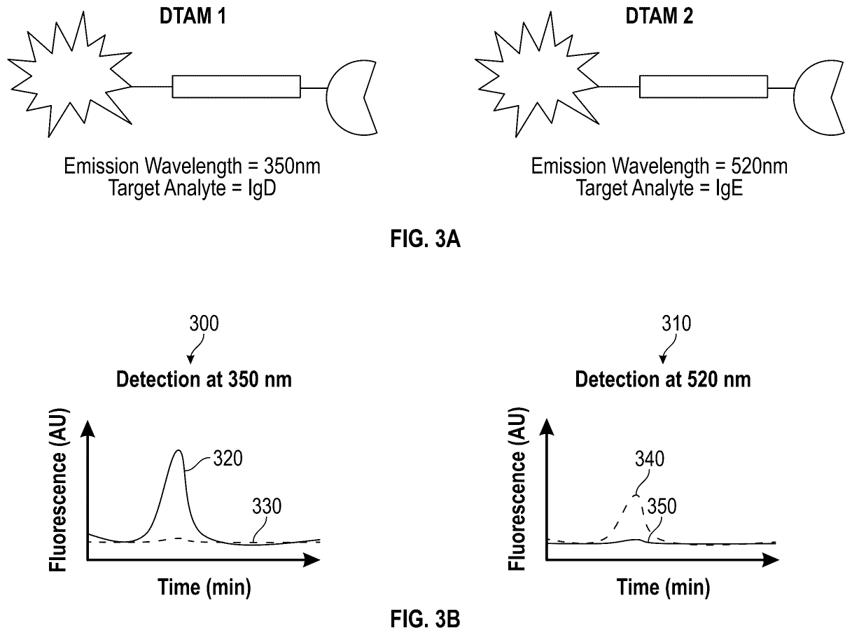

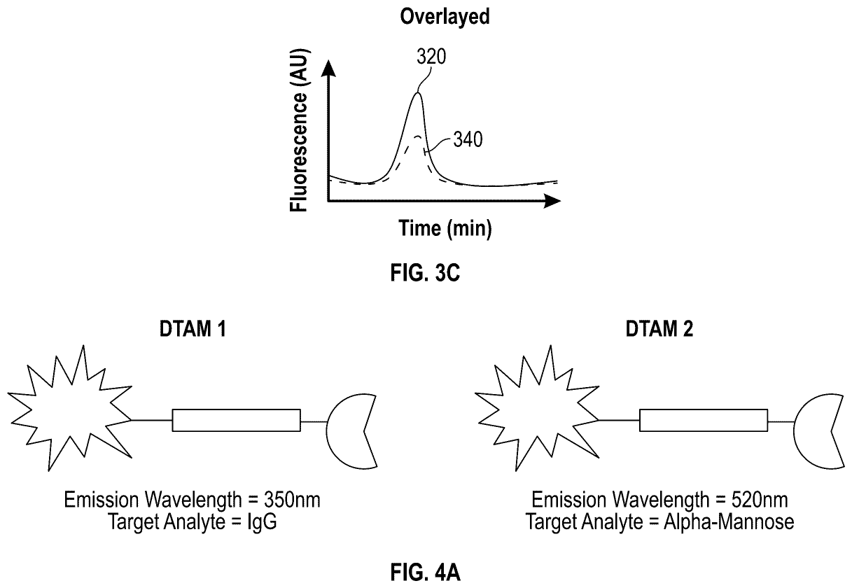

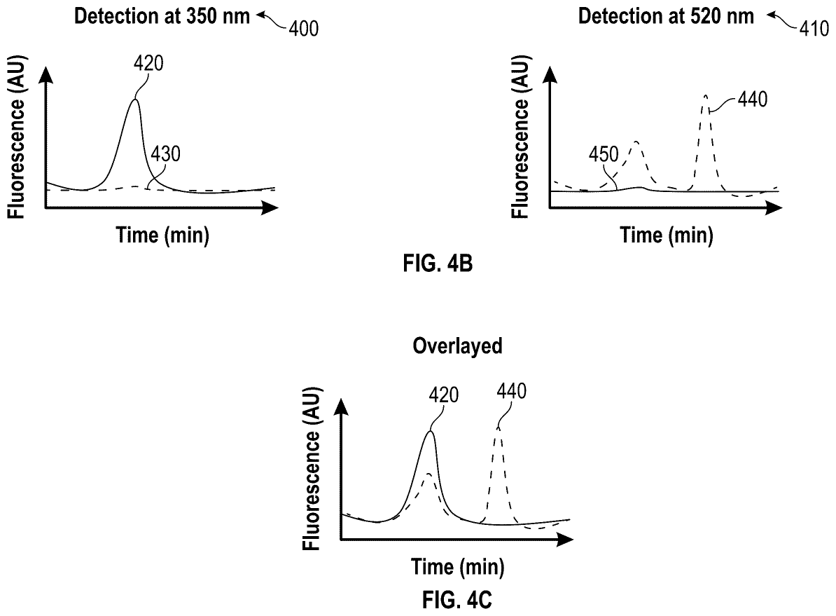

Using the methods disclosed herein, the attributes of a target analyte can be characterized. The detectors connected to the liquid chromatography system result in chromatograms, such as the UV chromatogram (210) and fluorescence chromatogram (230) of FIG. 2. Methods of analyzing and processing chromatograms produced from liquid chromatography assays are well known in the art, and as such a person of ordinary skill in the art would readily understand how to interpret the data generated from the methods disclosed herein. For the sake of clarity, two representative examples of a multiplexed assay are described and depicted in FIG. 3 (i.e., FIG. 3A, FIG. 3B, and FIG. 3C) and FIG. 4 (i.e., FIG. 4A, FIG. 4B, and FIG. 4C).

A representative assay for the detection and characterization of two unique analytes from a single assay is shown in FIG. 3. FIG. 3A depicts the two DTAMs utilized. DTAM 1 comprises an affinity group that specifically binds to IgD and a fluorescent dye with an emission wavelength of 350 nm. DTAM 2 comprises an affinity group that specifically binds to IgE and a fluorescent dye with an emission wavelength of 520 nm. Using the methods disclosed herein, fluorescence chromatograms are produced, which can be analyzed to determine relative quantities of IgD and IgE from a sample as shown in FIG. 3B. Chromatogram 300, detected at 350 nm, shows the relative fluorescence produced by DTAM 1 (320) and DTAM 2 (330). Chromatogram 310, detected at 520 nm, shows the relative fluorescence produced by DTAM 1 (350) and DTAM 2 (340). In this respect, the peak in the chromatogram for trace 320 correlates with the amount of IgD in the sample, and the peak in the chromatogram for trace 340 correlates with the amount of IgE in the sample. Since the DTAMs comprise compatible dyes, the DTAMs only produce background levels of fluorescence when detected outside their respective emission wavelengths. When the relevant traces for each DTAM are overlayed as shown in FIG. 3C, in this case traces 320 and 340, it is evident that IgE is at a lower concentration as compared to IgD. Thus, even though the IgD and IgE are similar in size (185 kDa vs 190 kDa, respectively), the relative concentration of the two antibodies from a single sample is detectable using a single assay in accordance with the methods disclosed herein.

A representative assay for the detection and characterization of multiple attributes directed to a single analyte is shown in FIG. 4. FIG. 4A depicts the two DTAMs utilized. DTAM 1 comprises an affinity group that specifically binds to IgG and a fluorescent dye with an emission wavelength of 350 nm. DTAM 2 comprises an affinity group that specifically binds to alpha-mannose and a fluorescent dye with an emission wavelength of 520 nm. Using the methods disclosed herein, fluorescence chromatograms are produced, which can be used to determine the amount of mannosylated-IgG from a sample as is shown in FIG. 4B. Chromatogram 400, detected at 350 nm, shows the relative fluorescence produced by DTAM 1 (420) and DTAM 2 (430). Chromatogram 410, detected at 520 nm, shows the relative fluorescence produced by DTAM 1 (450) and DTAM 2 (440). In this respect, the peak in the chromatogram for trace 420 correlates with the amount of IgG in the sample, and the peak in the chromatogram for trace 440 correlates with the amount of alpha-mannose in the sample. Since the DTAMs comprise compatible dyes, the DTAMs only produce background levels of fluorescence when detected outside their respective emission wavelengths. When the relevant traces for each DTAM are overlayed as shown in FIG. 4C, in this case traces 420 and 440, one can determine the amount of alpha-mannosylated IgG relative to non-alpha-mannosylated IgG.

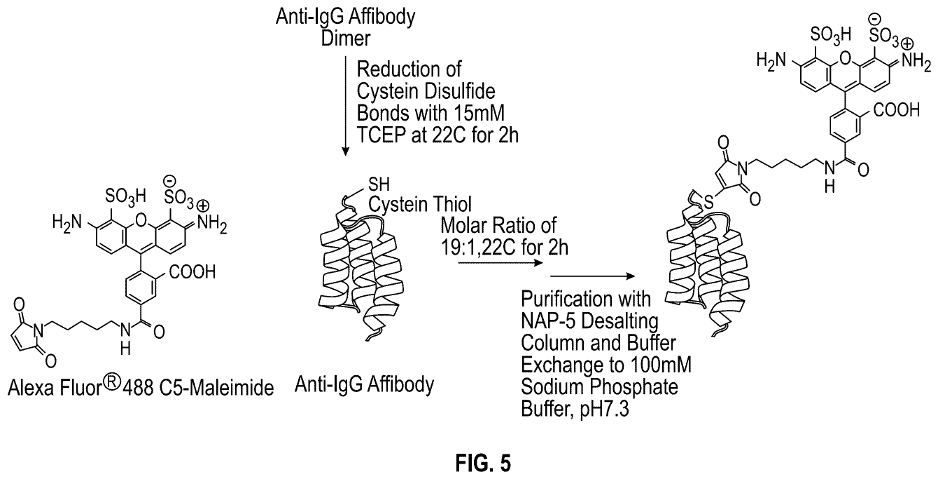

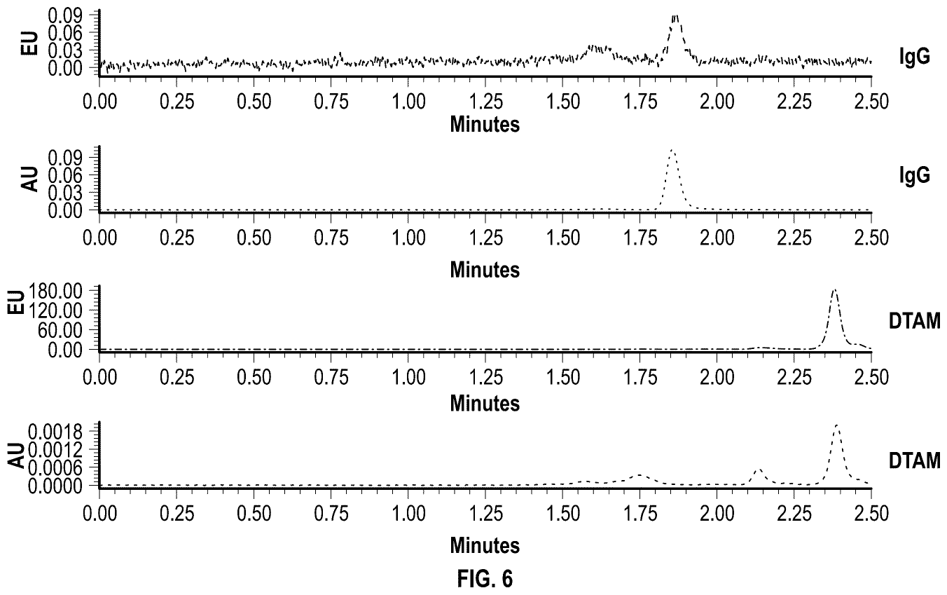

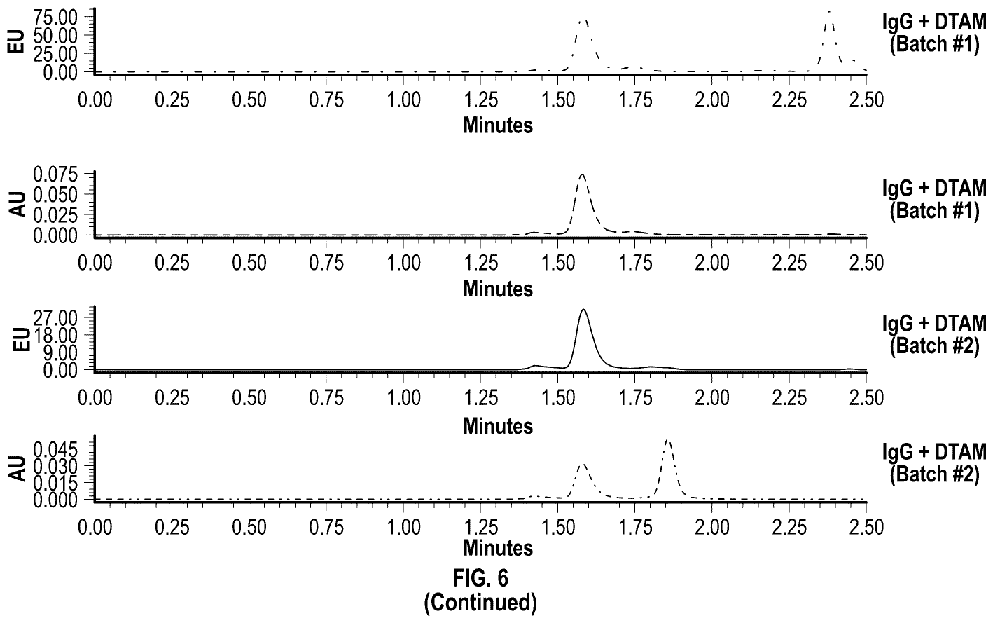

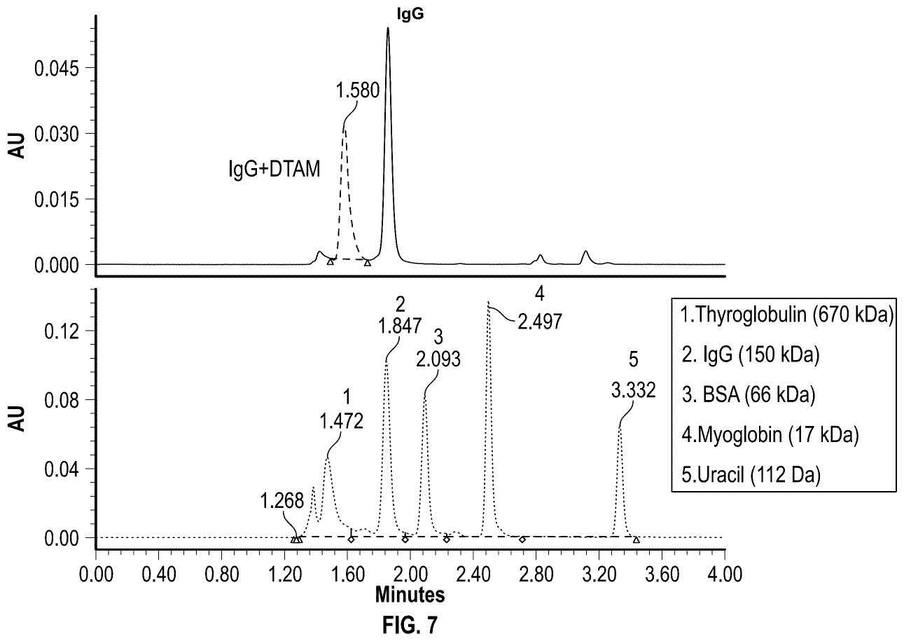

Example 1 and FIG. 5 provide an exemplary synthesis of DTAM described herein. Example 2 and FIG. 6 and FIG. 7 demonstrate the binding of an anti-IgG DTAM to IgG.

As one of skill in the art will appreciate, the column used in the liquid chromatography system will dictate the order in which the analytes elute from the column. For example, if a size-exclusion column is equipped, larger molecules will elute first. If a hydrophobic-interaction column is equipped, more hydrophilic molecules will elute first. The principles underlying liquid chromatography are well understood in the art, and as such one of ordinary skill in the art would readily understand how to interpret the resultant chromatograms.

### EXAMPLES

### Example 1: Synthesis of DTAMs

Synthesis of exemplary DTAMs is described. 5-FITC fluorescent dye (ThermoFisher Scientific) is conjugated to an anti-IgG affibody (Abcam) according to methods known in the art. The resultant DTAM is purified by desalting or spin columns to afford a purified FITC-affibody DTAM. The DTAM can be characterized by LC-MS and NMR. The above method can be repeated with tetramethylrhodamine-5-isothiocyanate, rhodamine-B-isothiocyanate, or NIR-797 isothiocyanate. The method can further be used with any dye and/or any affinity group disclosed herein. In some embodiments, the affinity group is an anti-IgG, anti-IgM, or anti-AAV antibody. In some embodiments, the affinity group is a lectin, including any lectin from the GlycoSelect series. In some embodiments, the affinity group is a lectin that binds to alpha-mannose.

One such synthesis of a DTAM is the conjugation of an Alexa Fluor® 488 C5-maleimide (available from Thermo Fisher Scientific) with an anti-IgG Affibody® (available from Affibody Medical AB). The anti-IgG Affibody® dimer was first reduced with 15 mM TCEP at 22° C. for 2 hours. The Alexa Fluor® 488 C5-maleimide dye was then incubated with the Affibody® at a molar ratio of 19:1 at 22° C. for 2 hours. The conjugate was then purified with a desalting column and further buffer exchanged to 100 mM sodium phosphate buffer (pH 7.3). The synthesis of the DTAM is shown in FIG. 5.

### Example 2: Characterization of a Target Analyte with One DTAM

An assay to characterize both titer and size of a target analyte is performed in this example. This example utilizes a control as the target analyte to illustrate the methods of the instant technology. A FITC-anti-IgG DTAM (such as those described in Example 1, ˜1 g/L or ˜1.3 equivalence to highest mAb concentration) is added to a vial (e.g., a polypropylene vial with a pre-silt PTFE/silicone septum, 300 μL) with a solution comprising humanized mAb Mass Check Standard (Waters Technologies Corporation, 0.1 to 10 g/L) and 200 mM ammonium acetate. The vial is capped and vortexed for ˜30 seconds prior to applying it to a liquid chromatography system equipped with a size-exclusion column, a UV, and a fluorescence detector. The sample (0.1 to 1 μL) is injected at a flow rate of 1 mL min in a 200 mM ammonium acetate mobile phase. In solution, the DTAM will selectively bind to IgG and be associated with IgG aggregates, monomeric IgG, and IgG fragments having the epitope to which the anti-IgG affibody binds. Excess DTAM will remain unbound in solution. Accordingly, the sample is first separated by size, prior to hitting the UV and fluorescence detectors (dual detectors). The peak area of the resultant chromatograms allows for the determination of antibody quantity, as well as the relative quantity of aggregate, monomer, fragment, or non-bound antibody. The fluorescence chromatogram can further be compared with a UV chromatogram for quality of analysis and to determine relative amounts of aggregate and monomer.

While the Example is described using a size-exclusion column, any column or separation technique that affords separation of a sample can be used. For example, a hydrophobic interaction, reversed-phase, normal phase, hydrophilic interaction, ion-exchange, mixed-mode, and/or slalom chromatography columns can be utilized. In addition, dialysis, analytical ultracentrifugation, and molecular weight filtration are suitable separation modes. Further, additional detectors as disclosed herein can be used in conjunction with the assay. For example, a MALS detector or a Charge Detection Mass Spectrometer (CDMS) can be used to gain additional particle characterization data such as size, shape, and molecular weight.

The above methods were used to characterize an IgG antibody (NISTmAb monoclonal IgG, available from Millipore Sigma). The IgG antibody sample was incubated with the DTAM as described in Example 1 (comprising the Alexa Fluor® 488 dye conjugated to the anti-IgG Affibody®) at one of two molar ratios: (i) a 5:1 ratio of DTAM to IgG (referred to as Batch #1) or (ii) a 1.21:1 ratio of DTAM to IgG (referred to as Batch #2). For both, the IgG was at a concentration of 1 μg/μL. Samples were incubated in 100 mM phosphate buffer (pH 7.3) for 2 h at 37° C.

Samples were analyzed on an ACQUITY™ Premier SEC column (available from Waters Technologies Corporation), namely a 4.6×150 mm column packed with particles having an average particle size of 1.7 μm and an average pore size of 250 Å. 20 μL of each sample was injected onto the column and flowed through using a 20 mM PBS mobile phase at a flow rate of 1 ml/min. FIG. 6 provides chromatograms for the IgG antibody sample alone (rows 1 and 2), the anti-IgG DTAM (rows 3 and 4), and the anti-IgG DTAM incubated with the IgG antibody for both Batch #1 (rows 5 and 6) and Batch #2 (rows 7 and 8). As shown in FIG. 6, the anti-IgG DTAM binds to the IgG sample, thereby altering the retention time on the SEC column. Comparing the elution times to a known standard confirms the binding of IgG with the DTAM as shown in FIG. 7.

### Example 3: Characterization of a Target Analyte with Two DTAMs

The assay of Example 2 can further be repeated with the addition of an Alexa Fluor 350-alpha-mannose-lectin (RPL-alpha-mannose; GlycoSelect) DTAM at approximately 15 g/L (˜5.2 equivalence to highest mAb concentration). The DTAMs comprise compatible dyes, with the FITC-anti-IgG DTAM having an emission wavelength of 520 nm and the Alexa Fluor 350-alpha-mannose-lectin DTAM having an emission wavelength of 442 nm.

The comparison between the traces in the fluorescence chromatograms provided by each DTAM permits the quantification of that to which the DTAM affinity group binds. In the context of this example, the assay can determine the quantity of both the IgG antibody, amount of alpha-mannose, and the amount of alpha-mannosylated-IgG antibodies. The fluorescence chromatogram can further be compared with a UV chromatogram for quality of analysis and to determine relative amounts of aggregate and monomer.

### Example 4: Characterization of a Target Analyte with Two DTAMs

The assay of Example 2 can further be repeated with the addition of an Alexa Fluor 680-Man2 lectin (RPL-mannose2; GlycoSelect) DTAM at approximately 15 g/L (˜5.2 equivalence to highest mAb concentration). The two DTAMs comprise compatible dyes, with the FITC-anti-IgG DTAM having an emission wavelength of 520 nm and the Alexa Fluor 680-Man2 lectin DTAM having an emission wavelength of 702 nm.

The comparison between the traces in the fluorescence chromatograms provided by each DTAM permits the quantification of that to which the DTAM affinity group binds. In the context of this example, the assay can determine the quantity of both the IgG antibody, amount of alpha-mannose, and the amount of alpha-mannosylated-IgG antibodies. The fluorescence chromatogram can further be compared with a UV chromatogram for quality of analysis and to determine relative amounts of aggregate and monomer.

### Example 4: Characterization of a Target Analyte with Three DTAMs

The assay of Example 3 or Example 4 can further be repeated with the addition of an Alexa Fluor 680-Man2 lectin (RPL-mannose2; GlycoSelect) DTAM or an Alexa Fluor 350-alpha-mannose-lectin (RPL-alpha-mannose; GlycoSelect) DTAM, respectively, at a concentration of approximately 15 g/L (˜5.2 equivalence to highest mAb concentration). The three DTAMs in combination have compatible dyes.

The fluorescence chromatogram can be compared to the UV chromatogram for quality of analysis and relative amounts of aggregate and monomer of IgG. The comparison of the fluorescence chromatograms allows for the characterization and quantitation of glycan structures relative to a separately run calibration curve.

### Example 5: Characterization of a Target Analyte in a Fluorimeter

The assays of Examples 2, 3, and 4 can be performed without chromatographic separation. Instead, sample comprising the DTAMs is added to a cuvette and measured using a fluorimeter and/or a UV-Vis spectrophotometer.

### Example 6: Evaluation of a DTAM for Use in Size Exclusion Chromatography

An assay to determine the suitability of a dye molecule of a DTAM for use in size exclusion chromatography (SEC) applications is described. A FITC-anti-IgG DTAM is added to a vial with a solution comprising uracil and 200 mM ammonium acetate without target analyte (in this example IgG). The vial is capped and vortexed for ˜30 seconds and then applied to a liquid chromatography system equipped with a size exclusion column, a UV detector, and a fluorescence detector. The sample (0.1 to 1 μL) is injected at a flow rate of 1 mL/min in a 200 mM ammonium acetate mobile phase. The retention time of the unbound DTAM is compared with the uracil marker and size calibration standards as monitored by both UV and fluorescence. Retention of a molecule that is not excluded from the pore of the SEC material can be associated to the particle volume and molecular size using calibration standards. Using an appropriately selected SEC material, the DTAM is not excluded from the pores of the SEC column material, and therefore the retention time of the DTAM should correspond to that of a particle of a similar size. An increase in retention time over uracil or the estimated retention time of the DTAM by its size indicates that the DTAM has undesirable interactions with the size-exclusion chromatography column. A significant reduction in retention time over the estimated elution time of the DTAM, or the observance of one or more peaks at a lower retention time than the peak observed for unbound DTAM, may indicate aggregation by size-exclusion chromatography.

The above assay can be used for a variety of DTAM molecules, including different dye molecules, linkers, and affinity groups.

1. A method for the simultaneous quantification and characterization of analytes in a composition, the method comprising:
a) selecting at least one dye-tagged affinity molecule (DTAM) of the formula D₁₋₈-L₀₋₈-M, wherein:
D represents an ultraviolet (UV) or a fluorescent dye molecule;
-L-represents an optionally substituted aliphatic, aromatic, heterocyclic, polyethylene glycol, carbohydrate, or peptide linker; and
M is an affinity group;
b) adding the at least one DTAM to a sample comprising at least one target analyte;
c) injecting the sample into a liquid chromatographic system comprising at least one detector; and
d) measuring at least one attribute of the at least one target analyte with the at least one detector.

2. The method of claim 1, wherein D is 1 and L is 1.

3. The method of claim 1, wherein the detector is selected from the group consisting of: UV, fluorescence, multi-angle light scattering, asymmetric flow field-flow fractionation, evaporative light scattering, refractive index, conductivity, charged aerosol, mass spectrometry, or charge detection mass spectrometry detector.

4. The method of claim 1, wherein the liquid chromatographic system comprises a size-exclusion column, reversed phase column, hydrophilic interaction column, hydrophobic interaction column, ion exchange column, chiral column, or combinations thereof.

5. The method of claim 1, wherein the affinity group is selected from the group consisting of: a polyclonal antibody, a monoclonal antibody, a fusion protein, a bispecific antibody, a chimeric antibody, a monobody, a nanobody, a camelid, an affimer, an affibody, an aptamer, a cyclodextrin, a peptide, DNA, RNA, a lectin, a chelating agent, a molecular imprinted polymer, or combinations thereof.

6. The method of claim 1, wherein the target analyte is a small molecule, a protein, a peptide, an antibody, a hormone, a pesticide, a toxin, and/or nucleic acids.

7. The method of claim 1, wherein the target analyte in the sample and the DTAM are at equivalent concentrations.

8. The method of claim 1, wherein the DTAM is at a higher concentration than the target analyte in the sample.

9. The method of claim 1, wherein the attribute is quantity, size, charge, post-translational modification, glycan concentration, and/or aggregation.

10. The method of claim 1, wherein more than one attribute is measured in parallel.

11. The method of claim 10, wherein the attributes measured in parallel are quantity, glycan concentration, and size.

12. The method of claim 10, wherein the attributes measured in parallel are quantity, glycan concentration, and charge.

13. The method of claim 11, wherein the quantity is antibody quantity and the glycan concentration is mannose, fucose, and/or a sialylated glycan concentration.

14. The method of claim 1, wherein two to six DTAMs are selected, wherein each of the two to six DTAMs have different emission wavelengths.

15. The method of claim 14, wherein two DTAMs are selected, wherein the affinity groups of each of the two DTAMs are an antibody.

16. The method of claim 14, wherein three DTAMs are selected, wherein the affinity groups of each of the three DTAMs are an antibody.

17. The method of claim 16, wherein the attributes measured in parallel are antibody quantity, mannose, fucose, and/or a sialylated glycan concentration, and size.

18. The method of claim 16, wherein the attributes measured in parallel are antibody quantity, mannose, fucose, and/or a sialylated glycan concentration, and charge.

19. The method of claim 14, wherein the DTAMs have affinity groups that bind to the same target analyte.

20. The method of claim 14, wherein the DTAMs have affinity groups that bind to unique target analytes.
